(19) United States
(12) Patent Application Publication
(10) Pub. No.: US 2009/0075273 A1
(43) Pub. Date: Mar. 19, 2009

(54) Planar Waveguide Detection Chips and Chambers for Performing Multiple PCR Assays

(75) Inventors: Ingmar Dorn, Koeln (DE); John J. Quinn, Madison, CT (US); Brian D. Warner, Martinez, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc., Tarrytown, NY (US)

(21) Appl. No.: 12/092,280

(22) PCT Filed: Nov. 9, 2006
(86) PCT No.: PCT/US06/60754

§ 371 Date: Aug. 12, 2008

Related U.S. Application Data
(60) Provisional application No. 60/736,925, filed on Nov. 14, 2005.

(51) Int. Cl. C12Q 1/68; C12M 1/34

(52) U.S. Cl. 435/6; 435/287.2

(57) ABSTRACT

Provided are planar waveguide (“PWG”) detection chips that are used to perform multiplex PCR and kinetic PCR assays with a single fluorescent dye. The PWG detection chips are housed in PWG detection chambers that house at least one PWG chip. The PWG detection chambers may be in a single chamber or a dual chamber configuration. Also provided are methods for analyzing amplification products using the PWG detection chambers of the present invention.

### TECHNICAL FIELD

The present invention relates generally to methods for performing multiplex PCR and kPCR assays. More specifically, the present invention relates to the use of planar waveguide (“PWG”) chips and detection chambers for performing multiplex PCR and kPCR assays using a single fluorescent dye.

### BACKGROUND OF THE INVENTION

Detection of biological molecules is essential to the fields of immunology, virology, molecular biological, genomics, proteomics, toxicology, forensics, drug screening, and clinical diagnostics. Most molecular detection systems detect biological molecules in an array format, which is analyzed via fluorescence excitation of the samples in the wells of the array.

As preferred technique for the detection and measurement of biological molecules is the polymerase chain reaction (“PCR”) assay, which quantitates amplification products of a nucleic acid analyte or gene by measuring the fluorescence of labeled dNTPs, and the kinetic PCR (“kPCR”) assay, which quantitates the amplification products by measuring the fluorescence of a reporter dye coupled to an amplification primer. Currently, no more than ten different analyte species can be simultaneously measured using either PCR or kPCR because no more than ten different fluorescent dyes can be used in a single PCR or kPCR assay to detect and measure individual amplification products. The present invention overcomes this shortcoming in the art by providing a method by which hundreds of analyte species may be analyzed in a single PCR or kPCR assay using a single fluorescent dye.

### SUMMARY OF THE INVENTION

To overcome the need in the art to screen multiple of analyte species in a single assay with a single dye, in one embodiment of the present invention, there is provided a method of performing a multiplex polymerase chain reaction (PCR) assay with a single fluorogenic dye comprising performing the PCR assay in a planar waveguide (PWG) incubation chamber.

In one aspect of the method, the PCR assay may be used to multiplex target genes, which may be selected from the group consisting of viral DNA, bacterial DNA, fungal DNA, and genomic DNA.

In another aspect of the method, the PCR assay is a reverse transcriptase PCR(RT-PCR) assay used to multiplex target RNAs, which may be selected from the group consisting of viral RNA, bacterial RNA, fungal RNA, or genomic RNA.

In yet another aspect of the method, the PCR assay is a kinetic PCR (kPCR) assay, which may be used to multiplex target genes, which may be selected from the group consisting of viral DNA, bacterial DNA, fungal DNA, and genomic DNA.

In a further aspect of the method, the PCR assay is a kinetic reverse transcriptase PCR (kRT-PCR) assay used to multiplex target RNAs, which may be selected from the group consisting of viral RNA, bacterial RNA, fungal RNA, or genomic RNA.

In another embodiment of the present invention, there is provided a planar waveguide (PWG) incubation chamber for performing polymerase chain reaction (PCR) assays, wherein the PWG incubation chamber houses at least one PWG chip.

In one embodiment of the chamber, the PWG incubation chamber has dual chambers comprised of an upper chamber and a lower chamber, wherein the upper and lower chambers are separated by a solid support and the at least one PWG chip is attached to the solid support on the upper chamber.

In one aspect of the dual chamber, the upper and lower chambers are covered with a flexible membrane, which may be molded with ridges on its underside.

In a further aspect of the dual chamber, the reaction mix for the PCR assay is pumped into the incubation chamber via a vacuum pump.

In still another aspect of the dual chamber, the reaction mix passes from the upper to the lower chambers via external ports.

In a further aspect of the dual chamber, the reaction mix circulates to the lower chamber and the flexible membrane on the upper chamber collapses. Where the flexible membrane has ridges, the ridges on the flexible membrane ensure that there is a distance of approximately 50 micron between the flexible membrane and the PWG chip surface.

In another embodiment of the chamber, the PWG incubation chamber is comprised of a single chamber on a solid support, wherein the PWG chip is attached to the solid support.

In one aspect of the single chamber, the chamber is covered with a flexible membrane, which may be molded with ridges on its underside.

In another aspect of the single chamber, the reaction mix is pumped into the chamber from a reservoir equipped with a pumping syringe.

In a further aspect of the single chamber, the reaction mix is pumped out of the single incubation chamber and the flexible membrane collapses. Where the flexible membrane has ridges, the ridges on the flexible membrane ensure that there is a distance of approximately 50 micron between the flexible membrane and the PWG chip surface. Additional aspects, advantages and features of the invention will be set forth, in part, in the description that follows, and, in part, will become apparent to those skilled in the art upon examination of the following, or may be learned by practice of the invention.

### BRIEF DESCRIPTION OF THE DRAWINGS

FIG. 1 schematically depicts a conventional PCR assay run on PWG chips. The amplicon being generated is rendered fluorescent via the incorporation of dNTPs labeled with a single fluorophore.

FIGS. 2A and 2B schematically depict a kPCR assay run on PWG chips. Universal capture overhangs on the degradable kPCR probes allow the probes to be captured at a specific location on the planar wavelength chip.

FIG. 3 schematically depicts a dual incubation chamber for housing PWG chips

FIG. 4 schematically depicts a single incubation chamber for housing PWG chips.

FIG. 5 provides a magnified view of the flexible membrane of the single incubation chamber of FIG. 4.

### DETAILED DESCRIPTION OF THE INVENTION

In describing and claiming the present invention, the following terminology will be used in accordance with the definitions set forth below.

As used in this specification and the appended claims, the singular forms “a,” “an,” and “the” include plural referents unless the context clearly dictates otherwise.

As used herein, the term “probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” refers to an oligonucleotide, whether produced naturally as in a purified restriction digest or produced synthetically, which is capable of acting as a point of initiation of synthesis when placed under conditions in which synthesis of a primer extension product that is complementary to a nucleic acid strand is induced, i.e., in the presence of appropriate nucleotides and an agent for polymerization such as a DNA polymerase in an appropriate buffer and at a suitable temperature. Within the context of the present invention, the term “amplification primer” refers to those primers used in target amplification procedures, such as PCR assays (which are geometric amplification reactions).

As used herein, the term “oligonucleotide” encompasses polydeoxyribonucleotides (containing 2-deoxy-D-ribose), polyribonucleotides (containing D-ribose), any other type of polynucleotide that is an N-glycoside of a purine or pyrimidine base, and other polymers containing nonnucleotidic backbones (e.g., protein nucleic acids and synthetic sequence-specific nucleic acid polymers commercially available from the Anti-Gene Development Group, Corvallis, Oregon, as NEUGENE™ polymers) or nonstandard linkages, providing that the polymers contain nucleobases in a configuration that allows for base pairing and base stacking, such as is found in DNA and RNA. Thus, “oligonucleotides” herein include double- and single-stranded DNA, as well as double- and single-stranded RNA and DNA:RNA hybrids, and also include known types of modified oligonucleotides, such as, for example, oligonucleotides wherein one or more of the naturally occurring nucleotides is substituted with an analog; oligonucleotides containing internucleotide modifications such as, for example, those with uncharged linkages (e.g., methyl phosphonates, phosphotriesters, phosphoramidates, carbamates, etc.), negatively charged linkages (e.g., phosphorothioates, phosphorodithioates, etc.), and positively charged linkages (e.g., aminoalkylphosphoramidates, aminoalkylphospbotriesters), those containing pendant moieties, such as, for example, proteins (including nucleases, toxins, antibodies, signal peptides, poly-L-lysine, etc.), those with interealators (e.g., acridine, psoralen, etc.), those containing chelators (e.g., metals, radioactive metals, boron, oxidative metals, etc.), and those containing alkylators. There is no intended distinction in length between the terms “polynucleotide” and “oligonucleotide,” and these terms will be used interchangeably. These terms refer only to the primary structure of the molecule. As used herein the symbols for nucleotides and polynucleotides are according to the IUPAC-IUBMB Joint Commission on Biochemical Nomenclature (see, http://www.chem.qmul.ac.uk/iupac/jcbn).

Oligonucleotides can be synthesized by known methods. Background references that relate generally to methods for synthesizing oligonucleotides include those related to 5′-to-3′ syntheses based on the use of β-cyanoethyl phosphate protecting groups. See, e.g., de Napoli et al., GAZZ CHIM ITAL 114:65 (1984); Rosenthal et al., TETRAHEDRON LETT 24:1691 (1983); Belagaje and Brush, NUC ACIDs RES 10:6295 (1977); in references which describe solution-phase 5′-to-3′ syntheses include Hayatsu and Khorana, J AM CHEM SOC 89:3880 (1957); Gait and Sheppard, NUC ACIDS RES 4:1135 (1977); Cramer and Koster, ANGEW CHEM INT ED ENGL 7:473 (1968); and Blackburn et al., J CHEM SOC PART C, at 2438 (1967). Additionally, Matteucci and Caruthers, J AM CHEM SOC 103:3185-91 (1981) describes the use of phosphochloridites in the preparation of oligonucleotides; Beaucage and Caruthers, TETRAHEDRON LETT 22:1859-62 (1981), and U.S. Pat. No. 4,415,732 to Caruthers et al. describe the use of phosphoramidites for the preparation of oligonucleotides. Smith, AM BIOTECH LAB, pp. 15-24 (December 1983) describes automated solid-phase oligodeoxyribonucleotide synthesis; and T. Horn and M. S. Urdea, DNA 5:421-25 (1986) describe phosphorylation of solid-supported DNA fragments using bis(cyanoethoxy)-N,N-diisopropylaminophosphine. See also, references cited in Smith, supia; Warner et al., DNA 3:401-11 (1984); and T. Hom and M. S. Urdea, TETRAHEDRON LETT 27:4705-08 (1986).

The terms “nucleotide” and “nucleoside” refer to nucleosides and nucleotides containing not only the four natural DNA nucleotidic bases, i.e., the purine bases guanine (G) and adenine (A) and the pyrimidine bases cytosine (C) and thymine (T), but also the RNA purine base uracil (U), the non-natural nucleotide bases iso-U and iso-C, universal bases, degenerate bases, and other modified nucleotides and nucleosides. Universal bases are bases that exhibit the ability to replace any of the four normal bases without significantly affecting either melting behavior of the duplexes or the functional biochemical utility of the oligonucleotide. Examples of universal bases include 3-nitropyrrole and 4-, 5-, and 6-nitroindole, and 2-deoxyinosine (dl), that latter considered the only “natural” universal base. While dI can theoretically bind to all of the natural bases, it codes primarily as G. Degenerate bases consist of the pyrimidine derivative 6H,8H-3,4-dihydropyrimido[4,5-c][1,2]oxazin-7-one (P), which when introduced into oligonucleotides base pairs with either G or A, and the purine derivative N6-methoxy-2,6,-diaminopurine (K), which when introduced into oligonucleotides base pairs with either C or T. Examples of the P and K base pairs include P-imino, P-amino, K-imino, and K-amino.

Modifications to nucleotides and nucleosides include, but arc not limited to, methylation or acylation of purine or pyrimidine moieties, substitution of a different heterocyclic ring structure for a pyrimidine ring or for one or both rings in the purine ring system, and protection of one or more functionalities, e.g., using a protecting group such as acetyl, difluoroacetyl, trifluoroacetyl, isobutryl, benzoyl, and the like. Modified nucleosides and nucleotides also include modifications on the sugar moiety, e.g., wherein one or more of the hydroxyl groups are replaced with halide and/or hydrocarbyl substituents (typically aliphatic groups, in the latter case), or are functionalized as ethers, amines, or the like. Examples of modified nucleotides and nucleosides include, bat are not limited to, 1-methyladenine, 2-methyl adenine, N⁶-methyladenine, NW-isopentyl-adenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methyl-aminomethyl)uracil, 5-(carboxymethylaminomethyl)-uracil, 2-thiouracil, 5-methyl-2-thiouracil, S-(2-broniovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine, and 2,6-diaminopurine.

As used herein, the term “nucleic acid” refers to polynucleotides such as deoxyribonucleic acid (DNA), and, where appropriate, ribonucleic acid (RNA). The term should also be understood to include, as equivalents, analogs of RNA or DNA made from nucleotide analogs, and, as applicable to the embodiment being described, single (sense or antisense) and double-stranded polynucleotides. Expressed Sequence Tags (“ESTs,” i.e., small pieces of DNA sequence usually 200 to 500 nucleotides long generated by sequencing either one or both ends of an expressed gene), chromosomes, cDNAs, mRNAs, and rRNAs are representative examples of molecules that may be referred to as nucleic acids.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer. Such minor modifications of the disclosed sequences and any necessary adjustments of hybridization conditions to maintain specificity require only routine experimentation and are within the ordinary skill in the art.

The term “hybridizing conditions” is intended to mean those conditions of time, temperature, and ply, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation. Typical hybridizing conditions include the use of solutions buffered to a pH from about 7 to about 8.5 and temperatures of from about 30° C. to about 60° C., preferably from about 37° C. to about 55° C. for a time period of from about one second to about one day, preferably from about 15 minutes to about 16 hours, and most preferably from about 15 minutes to about three hours. Hybridization conditions should also include a buffer that is compatible, i.e., chemically inert, with respect to primers, probes, and other components, while still allowing for hybridization between complementary base pairs. The selection of such buffers is within the knowledge of one of ordinary skill in the art.

It is understood by one of ordinary skill in the art that the isolation of DNA and RNA target sequences from a sample requires different hybridization conditions. For example, if the sample is initially disrupted in an alkaline buffer, double stranded DNA is denatured and RNA is destroyed. By contrast, if the sample is harvested in a neutral buffer with SDS and proteinase K, DNA remains double stranded and cannot hybridize with the primers and/or probes and the RNA is protected from degradation.

The terms “support” and “substrate” are used interchangeably to refer to any solid or semi-solid surface to which an oligonucleotide probe or primer, analyte molecule, or other chemical entity may be anchored. Suitable support materials include, but are not limited to supports that are typically used for solid phase chemical synthesis such as polymeric materials and plastics for use in beads, sheets, and microtiter wells or plates examples including without limitation, polystyrene, polystyrene latex, polyvinyl chloride, polyvinylidene fluoride, polyvinyl acetate, polyvinyl pyrrolidone, polyacrylonitrile, polyacrylamide, polymethyl methacrylate, polytetrafluoroethylene, polyethylene, polypropylene, polycarbonate, and divinylbenzene styrene-based polymers; polymer gels; agaroses such as SEPHAROSE®; dextrans such as SEPHADEX®); celluloses such as nitrocellulose; cellulosic polymers; polysaccharides; silica and silica-based materials; glass (particularly controlled pore glass) and functionalized glasses; ceramics, and metals. Preferred supports are solid substrates in the form of beads or particles, including microspheres, nanospheres, microparticles, and nanoparticles.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms (particularly ³²P and ¹²⁵I), electron-dense reagents, enzymes, and ligands having specific binding partners. Enzymes are typically detected by their activity. Within the context of flow cytometric analysis, preferred labels include biotinylated primary agents (such as biotinylated dNTPs) that hybridized to a target (such as an amplification sequence from a PCR) and streptavidin-phycoerythrin (“SA-PE”) as secondary agents, where the streptavidin acts as a developer by binding to the biotinylated primary agent and the phycoerythrin acts as the stain.

As used herein, the term “target amplification” refers to enzyme-mediated procedures that are capable of producing billions of copies of nucleic acid target. Examples of enzyme-mediated target amplification procedures known in the art include PCR, nucleic acid-sequence-based amplification (“NASBA”), transcription-mediated amplification (“TMA”), strand displacement amplification (“SDA”), and ligase chain reaction (“LCR”).

The most widely used target amplification procedure is PCR, first described for the amplification of DNA by Mullins et al. in U.S. Pat. No. 4,683,195 and Mullis in U.S. Pat. No. 4,683,202 (also referred to herein as the “conventional PCR assay”). In the PCR assay, a sample of DNA is mixed in a solution with a molar excess of two oligonucleotide primers of 10-30 base pairs each that are prepared to be complementary to the 3′ end of each strand of the DNA duplex; a molar excess of unattached nucleotide bases (i.e., dNTPs); and DNA polymerase, (preferably Taq polymerase, which is stable to heat), which catalyzes the formation of DNA from the oligonucleotide primers and dNTPs. Where desired, the dNTPs may be labeled with a fluorescent dye. Of the two primers, one is a forward primer that will bind in the 5′-3′ direction to the 3′ end of one strand of the denatured DNA analyte and the other is a reverse primer that will bind in the 3′-5′ direction to the 3′ end of the other strand of the denatured DNA analyte. The solution is heated to 94-96° C. to denature the double-stranded DNA to single-stranded DNA. When the solution cools, the primers bind to the separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. In other words, an extension product synthesized from the forward primer, upon separation, would serve as a template for a complementary extension product synthesized from the reverse primer. Similarly, the extension product synthesized from the reverse primer, upon separation, would serve as a template for a complementary extension product synthesized from the forward primer. In this way, the region of DNA between the primers is selectively replicated with each repetition of the process. Since the sequence being amplified doubles after each cycle, a theoretical amplification of one billion copies may be attained after repeating the process for a few hours; accordingly, extremely small quantities of DNA may be amplified using PCR in a relatively short period of time.

Where the starting material for the PCR assay is RNA, complementary DNA (“cDNA”) is made from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. The enzymes are used in genetic engineering to produce specific cDNA molecules from purified preparations of mRNA. A PCR used to amplify RNA products is referred to as reverse transcriptase PCR or “RT-PCR.”

The terms “kinetic PCR” (“kPCR”) or “kinetic RT-PCR” (“kRT-PCR”), which are also referred to as “real-time PCR” and “real-time RT-PCR,” refer to the detection of PCR products via a fluorescent signal generated by the coupling of a fluorescent reporter dye (also referred to herein as a “fluorophore” or “fluorescent dye”) and a quencher moiety to the same or different oligonucleotide substrates. Examples of commonly used probes used in kPCR and kRT-PCR include the following probes: Taqman® probes, Molecular Beacons probes, Scorpions® probes, and SYBR® Green probes. Briefly, Taqman probes, Molecular Beacons, and Scorpion probes each have a fluorophore attached to the 5′ end of the probes and a quencher moiety coupled to the 3′ end of the probes. In the unhybridized state, the proximity of the fluorophore and the quencher moiety prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green probes binds double-stranded DNA and upon excitation emit light; thus as PCR product accumulates, fluorescence increases.

When the term “PCR assays” is used generally herein, it is used to describe all the PCR assays described above, that is, the term is meant to include the conventional PCR assay first described by Mullins and Mullins et al., RT-PCR, kPCR, and kRT-PCR.

The terns “amplification sequence,” “amplification product,” “PCR product,” “PCR amplification product,” and “amplicon(s)” all refer to the single-stranded sequences that are the end product of PCR assays.

The term “singleplex” refers to a single assay that is not carried out simultaneously with any other assays. Singleplex assays include individual assays that are carried out sequentially.

The term “multiplex” refers to multiple assays that are carried out simultaneously, in which detection and analysis steps are generally performed in parallel. As used herein, a multiplex assay may also be termed according to the number of target sites that the assay aims to identify. For example, a multiplex assay that is designed to identify twenty PCR products may be referenced a “twentyplex” assay and a multiplex assay that is designed to identify fifty PCR products may be referenced a “fiftyplex” assay.

The following description of the preferred embodiments and examples are provided by way of explanation and illustration and are not to be viewed as limiting the scope of the invention as defined by the claims. Any alternates or modifications in function, purpose, or structure are intended to be covered by the claims of this application.

Performing PCR Assays on PWG Chips

As previously noted, conventional PCR assays measure amplification products in a 1:1 ratio of analyte:fluorophore; thus, using conventional PCR, in order to measure multiple analytes in a sample, ten different fluorescent dyes must be used and measured in the assay. The present invention overcomes this shortcoming in PCR technology by capturing amplification products kinetically during the thermocycling process onto a two dimensional surface where many different species can be multiplexed and measured using a single fluorescent dye. The two-dimensional surface that is used to achieve the kPCR assay of the present invention is a planar waveguide (“PWG”) chip.

PWG technology combines highly selective fluorescence detection with high sensitivity. PWGs are 150 to 300 nm thin films made of a material with a high refractive index, such as titanium dioxide (TiO₂) or tantalum pentoxide (Ta₂O₅), that arc deposited on a transparent support with a low refractive index, such as glass, silicon dioxide, or a polymer. A parallel laser light beam is coupled into the waveguiding film by a diffractive grating that is etched or embossed into the substrate. When the light propagates within the film, a strong evanescent field that is perpendicular to the direction of propagation is produced, which enters into the adjacent medium. The intensity of he evanescent filed can be enhanced by increasing the refractive index of the waveguiding layer and decreasing the layer thickness. Compared to confocal excitation of the field intensity close to the surface can be increased by a factor or up to 100. The field strength decays exponentially with the distance from the waveguide surface and its penetration depth is limited to about 400 nm. his effect can be used to selective excite only fluorophores located at or near the surface of the waveguide, which in turn results in a significant decrease in background interference that results from fluorescence emission of the solution in the well.

When PWG technology is used for bioanalytic applications, specific capture probes or recognition elements for the analyte of interest are immobilized on the PWG surface. The presence of the analyte in a sample applied to a PWG chip is detected using fluorophores attached to the analyte or one of its binding partners in the assay. Upon fluorescence excitation of the evanescent field, excitation and detection of fluorophores is restricted to the sensing surface, while signals from unbound molecules in the bulk solution are not detected. The result is an increase in the signal to noise ratio in microarrays over conventional optical detection methods. Further, as only a fraction of the amplicons in a reaction mixture would need to be captured during each cycle of a PCR assay in order to obtain a reading, the speed of the PCR analysis remains in synchronicity with the overall thermocycling process of the PCR assay.

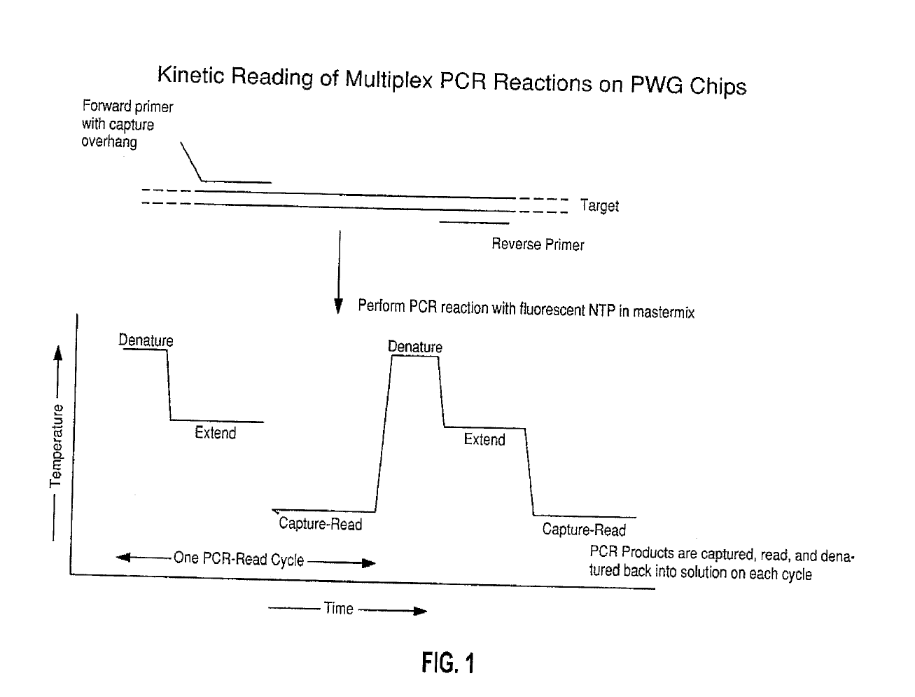

In one embodiment of the present invention, which is depicted schematically in FIG. 1, a multiplex conventional PCR assay is performed on PWG chips. As shown in FIG. 1, forward and reverse primers are prepared for multiple nucleic acid sequences with the forward primer having a unique capture sequence overhang for each target nucleic acid sequence; the unique capture sequence overhangs will hybridize to a complementary unique tag sequences that arc attached to the surface of the PWG chips. The PCR mastermix includes fluorescent dNTPs that produce the multiple PCR extension products. Since the PWG chips only measures label bound to the tag sequence on the surface of the PWG chips, free dNTPs should contribute minimal background. The PWG format allows multiple PCR products to be monitored, potentially many more than with kPCR assays that use multiple fluorescent dyes.

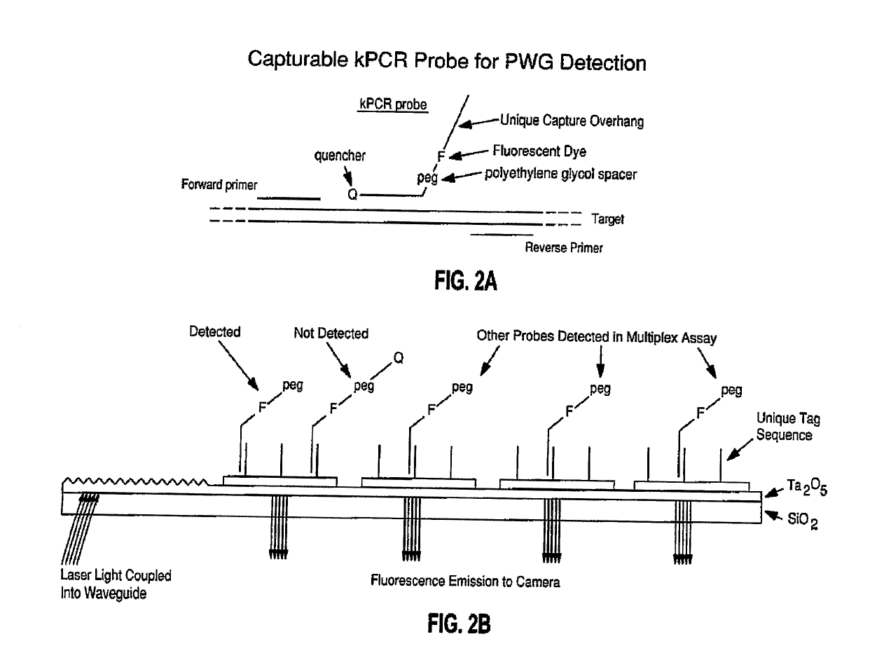

In another embodiment of the present invention, which is depicted schematically in FIGS. 2A and 2B, a kPCR reaction is preformed on PWG chips. As shown in FIG. 2A, the fluorescent kPCR probes include polyethylene glycol (“PEG”) spacers between the quencher moiety and the fluorescent dye; the PEG spacers serve to prevent nuclease degradation of the kPCR probes and the capture probes during the thermocycling process. As shown in FIGS. 2A and 2B, unique capture overhang sequences on the fluorescent dyes direct the kPCR probes to hybridize to complementary unique tag sequences that are attached at specific locations on the PWG chip surface. As indicated in FIG. 2B, the kPCR probes are hydrolyzed during the forward polymerization process by the exonuclease activity of the polymerase such that only hydrolyzed, i.e., degraded, kPCR probes are detected.

The PWG chips of the present invention may be housed in incubation chambers that are designed to accelerate the transport of the analytes to the PWG chip surface and consequently, ensure timely measurement of the analytes during the PCR thermocycling process. By rapidly and repeatedly cycling the assay solution through the device, analyte molecules in the solution are cycled to the surface of the PWG chip for capture.

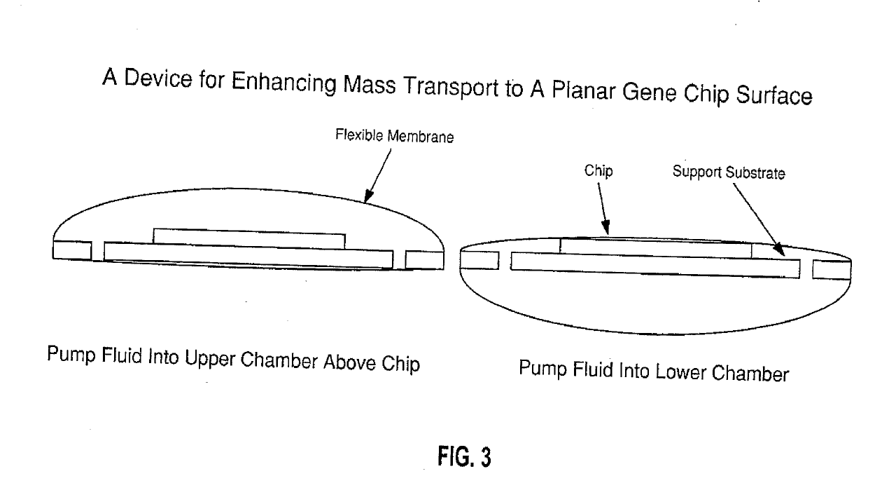

In one embodiment of the incubation chamber, which is depicted schematically in FIG. 3, the device consists of two chambers, an upper chamber and a lower chamber, which are individually encased by two flexible polymeric membranes (one for the upper chamber and the other for the lower chamber) that are bonded to a solid substrate that holds the PWG chip. The solid support substrate is constructed with ports allowing liquid to pass between chambers. A vacuum is applied to the outside of the device either above or below the chip to force liquid to migrate from chamber to chamber. When the flexible membrane is brought into close contact with the chip surface, it conforms to the chip due to its flexibility. Preferably, the interior surface of the flexible polymer membrane is textured to ensure that a 50-micron thick layer of fluid is trapped at the surface thereby minimizing the diffusion distance for the solution The pumping cycle is repeated multiple times to mix the bulk solution while enhancing transport to the chip surface. Reagent solutions are introduced into the device through external ports in the substrate.

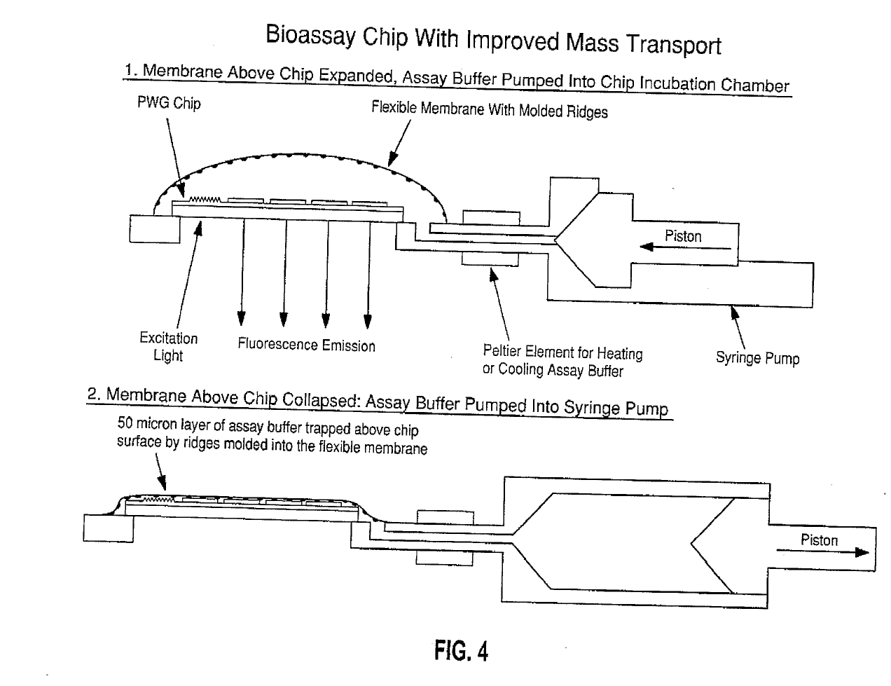

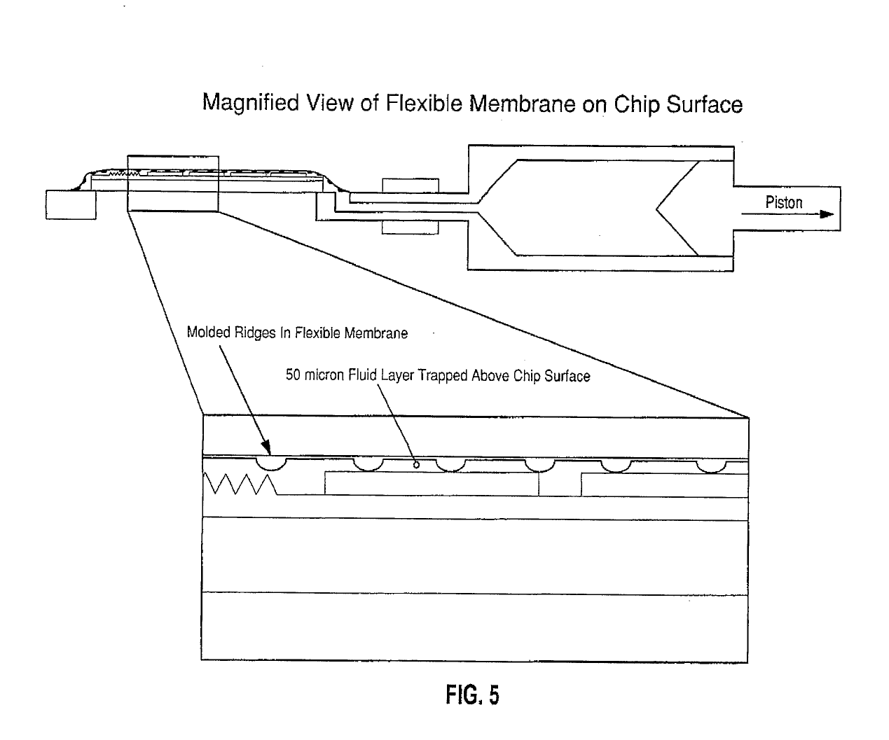

In another embodiment of the incubation chamber, which is depicted schematically in FIG. 4, the device consists of a single chamber encased by a flexible polymeric membrane that houses the PWG chip In this embodiment, the assay solution is pushed into and out of the chamber using a syringe pump, which houses a piston. Between the chamber and the syringe pump, it is possible to include a Peltier element that may heat or cool the assay solution. In addition, to ensure a 50-micron layer between the flexible membrane and the PWG chip surface, the flexible membrane may include molded ridges an its underside; such molded ridges may also be added to the underside of the flexible membrane of the incubation chamber depicted in FIG. 3. FIG. 5 schematically shows a magnified view of the incubation chamber of FIG. 4 when the PWG chip surface is in contact with the molded ridges of the flexible membrane.

As previously discussed, the present invention has the advantage of being capable of detecting multiple PCR products in a single kPCR assay run with a single fluorescent dye. For example, the kPCR assay of the present invention may be used to multiplex DNA and RNA for the presence of infectious agents, mRNA gene products, or DNA amplifications associated with the occurrence of cancer.

It is to be understood that while the invention has been described in conjunction with the preferred specific embodiments thereof, that the foregoing description as well as the examples that follow are intended to illustrate and not limit the scope of the invention. Other aspects, advantages, and modifications within the scope of the invention will be apparent to those skilled in the art to which the invention pertains. All publications mentioned herein, both supra and infra, are incorporated by reference in their entireties.

The following examples are put forth so as to provide those of ordinary skill in the art with a complete disclosure and description of how to make and use the compositions of the invention. The examples are intended as non-limiting examples of the invention. Efforts have been made to ensure accuracy with respect to numbers (e.g., amounts, temperature, etc.) but some experimental error and deviations should, of course, be allowed for, Unless indicated otherwise, parts are parts by weight, temperature is degrees centigrade and pressure is at or near atmospheric. All components were obtained commercially unless otherwise indicated.

### EXPERIMENTAL

In the examples that follow, efforts have been made to ensure accuracy with respect to numbers (e.g., amounts, temperature, etc.), but experimental error and deviations should be taken into account when conducting the described experiments. Unless indicated otherwise, parts are parts by weight, temperature is degrees centigrade, and pressure is at or near atmospheric. Unless otherwise indicated, all components described in the following examples were obtained through commercial sources.

### Example 1

### Viral Multiplex PCR Assay Performed on PWG Chips

Forward and reverse primers are constructed for a PCR assay that is to detect DNA sequences from 20 different viral genes, the forward primers having a unique universal capture overhang sequence to correspond to each of the 20 different viral genes. The viral gene sequences are obtained from publicly available databases.

A PWG chip is prepared by depositing a 200 nm film of titanium dioxide (TiO₂) on a silicon dioxide support substrate. A laser light is coupled into the PWG chip. Immobilized on the PWG surface are 20 different capture probes with unique tag sequences that are designed to hybridize to the individual unique universal capture overhangs on the forward primers.

The PCR primers are placed in the PCR mastermix along with the target DNA sequences and the dNTPs labeled with a single fluorescent dye. The PCR assay is run in a dual chamber planar chip incubation chamber (FIG. 3). When the universal sequences on the forward primers hybridize to the tag sequences on the capture probes, the fluorophores on the PCR extension products for each of the 20 viral genes produce a fluorescence emission from the PWG chip to a camera for analysis.

### Example 2

### Viral kPCR Multiplex Assay Performed on PWG Chips

Forward and reverse primers are constructed for a kPCR assay that is to detect DNA sequences from 20 different viral genes. The viral gene sequences are obtained from publicly available databases.

Kinetic PCR capture probes are constructed to include a quencher moiety, an oligonucleotide sequence that corresponds to a complementary segment of the viral sequences, a PEG spacer, a single fluorescent reporter dye, and a unique capture overhang sequence to correspond to each of the 20 different viral genes.

A PWG chip is prepared by depositing a 200 nm film of tantalum pentoxide (Ta₂O₅) on a silicon dioxide support substrate. A laser light is coupled into the PWG chip. Immobilized on the PWG surface are 20 different capture probes with unique tag sequences that are designed to hybridize to the individual unique capture overhangs on the kPCR probe.

The kPCR primers are placed in the kPCR mastermix along with the target DNA sequences, the fluorescent kPCR probes, and unlabeled dNTPs. The kPCR assay is run in a dual chamber planar chip incubation chamber (FIG. 3). When the polymerization is complete and the reporter dye is released, the unique capture overhang sequences attached to the reporter dye hybridize to the complementary tag sequences on the PWG chips and the fluorescence emission from the PWG chips is transmitted to a camera for analysis. Because there are 20 different capture overhang sequences and 20 different tag sequences that correspond to each of the 20 viral genes in the multiplex assay, quantitative measurements may be obtained for each of the 20 different viral genes.

### Example 3

### mRNA Oncogene PCR Multiplex Assay Performed on PWG Chips

Forward and reverse primers are constructed for a RT-PCR assay that is used to detect single-stranded sequences obtained from 50 different mRNA oncogenes sequences, the forward primers having a unique universal capture overhang sequence to correspond to each of the 50 different oncogenes. The oncogene mRNA gene sequences are obtained from publicly available databases.

A PWG chip is prepared by depositing a 200 nm film of titanium dioxide (TiO₂) on a silicon dioxide support substrate. A laser light is coupled into the PWG chip. Immobilized on the PWG surface are 50 different capture probes with unique tag sequences that are designed to hybridize to the individual unique universal capture overhangs on the forward primers.

The PCR primers are placed in the PCR mastermix along with the target sequences and the dNTPs labeled with a single fluorescent dye. The PCR assay is run in a dual chamber planar chip incubation chamber (FIG. 3). When the universal sequences on the forward primers hybridize to the tag sequences on the capture probes, the fluorophores on the PCR extension products for each of the 50 oncogene sequences produce a fluorescence emission from the PWG chip to a camera for analysis.

### Example 4

### mRNA Oncogene kPCR Multiplex Assay Performed on PWG Chips

Forward and reverse primers are constructed for a kRT-PCR assay that is used to detect single-stranded sequences obtained from 50 different mRNA oncogene sequences. The oncogene mRNA gene sequences are obtained from publicly available databases.

Kinetic PCR capture probes are constructed to include a quencher moiety, an oligonucleotide sequence that corresponds to a complementary segment of the oncogene sequences, a PEG spacer, a single fluorescent reporter dye, and a unique capture overhang sequence to correspond to each of the 20 different viral genes.

A PWG chip is prepared by depositing a 200 nm film of tantalum pentoxide (Ta₂O₅) on a silicon dioxide support substrate. A laser light is coupled into the PWG chip. Immobilized on the PWG surface are 50 different capture probes with unique tag sequences that are designed to hybridize to the individual unique capture overhangs on the kPCR probe.

The kPCR primers are placed in the kPCR mastermix along with the target sequences, the fluorescent kPCR probes, and unlabeled dNTPs. The kPCR assay is run in a dual chamber planar chip incubation chamber (FIG. 3). When the polymerization is complete and the reporter dye is released, the unique capture overhang sequences attached to the reporter dye hybridize to the complementary tag sequences on the PWG chips and the fluorescence emission from the PWG chips is transmitted to a camera for analysis. Because there are 50 different capture overhang sequences and 50 different tag sequences that correspond to each of the 50 oncogene sequences in the multiplex assay, quantitative measurements may be obtained for each of the 50 different oncogene sequences.

We claim:

1. A method of performing a multiplex polymerase chain reaction (PCR) assay with a single fluorogenic dye comprising performing the PCR assay in a planar waveguide (PWG) incubation chamber.

2. The method of claim 1, wherein the PCR assay is used to multiplex target genes.

3. The method of claim 2, wherein the target genes are selected from the group consisting of viral DNA, bacterial DNA, fungal DNA, and genomic DNA.

4. The method of claim 1, wherein the PCR assay is a reverse transcriptase PCR(RT-PCR) assay used to multiplex target RNAs.

5. The method of claim 4, wherein the target RNAs are selected from the group consisting of viral RNA, bacterial RNA, fungal RNA, or genomic RNA.

6. The method of claim 5, wherein the PCR assay is a kinetic PCR (kPCR) assay.

7. The method of claim 6, wherein the kPCR assay is used to multiplex target genes.

8. The method of claim 7, wherein the target genes are selected from the group consisting of viral DNA, bacterial DNA, fungal DNA, and genomic DNA.

9. The method of claim 5, wherein the PCR assay is a kinetic reverse transcriptase PCR (kRT-PCR) assay used to multiplex target RNAs.

10. The method of claim 9, wherein the target RNAs are selected from the group consisting of viral RNA, bacterial RNA, fungal RNA, or genomic RNA.

11. A planar waveguide (PWG) incubation chamber for performing polymerase chain reaction (PCR) assays, wherein the PWG incubation chamber houses at least one PWG chip.

12. The method of claim 11, wherein the PWG incubation chamber is comprised of an upper chamber and a lower chamber, wherein the upper and lower chambers are separated by a solid support and the at least one PWG chip is attached to the solid support on the upper chamber.

13. The method of claim 12, wherein the upper and lower chambers are covered with a flexible membrane.

14. The method of claim 13, wherein reaction mix for the PCR assay is pumped into the incubation chamber via a vacuum pump.

15. The method of claim 14, wherein reaction mix passes from the upper to the lower chambers via external ports.

16. The method of claim 15, wherein the flexible membrane is molded with ridges on its underside.

17. The method of claim 16, wherein when the reaction mix circulates to the lower chamber and the flexible membrane on the upper chamber collapses, the ridges on the flexible membrane ensure that there is a distance of approximately 50 micron between the flexible membrane and the PWG chip surface.

18. The method of claim 11, wherein the PWG incubation chamber is comprised of a single chamber on a solid support, wherein the PWG chip is attached to the solid support.

19. The method of claim 18, wherein the chamber is covered with a flexible membrane.

20. The method of claim 19, wherein reaction mix is pumped into the chamber from a reservoir equipped with a pumping syringe.

21. The method of claim 20, wherein the flexible membrane is molded with ridges at its underside.

22. The method of claim 21, wherein when reaction mix is pumped out of the single incubation chamber and the flexible membrane collapses, the ridges on the flexible membrane ensure that there is a distance of approximately 50 micron between the flexible membrane and the PWG chip surface.
